(19) United States
(12) Patent Application Publication
(10) Pub. No.: US 2007/0154432 A1
(43) Pub. Date: Jul. 5, 2007

(54) Compositions and methods for hair growth

(76) Inventor: Rose Davis, Harbor City, CA (US)

Correspondence Address: Rutan & Tucker, LLP.;Hani Z. Sayed, 611 ANTON BLVD, SUITE 1400, COSTA MESA, CA, 92626, US

(21) Appl. No.: 11/473,970

(22) Filed: Jun. 23, 2006

Related U.S. Application Data
(60) Provisional application No. 60/693,540, filed on Jun. 24, 2005.

(51) Int. Cl. A61K 31/4166; A61K 8/37; A61K 8/89; A61K 33/34; A61K 33/32

(52) U.S. Cl. 424/70.12; 424/70.31; 514/398; 424/642; 424/638

(57) ABSTRACT

A composition that promotes hair growth includes a lipophilic compound, such as a fatty acid, an allantoin, and a therapeutically effective amount of a biologically active substance. The fatty acid can be a palmitic, palmitoleic, stearic, oleic, linoleic, linolenic, arachidic, eicosenoic, behenic, erucic, myristic acid, and lignoderic fatty acid. The composition also includes jojoba oil, tea tree oil and wild carrot seed oil. A method for promoting hair growth using such composition.

This application claims priority to U.S. provisional application Ser. No. 60/693540 filed Jun. 24, 2005.

### FIELD OF THE INVENTION

The field of the invention is for promotion of hair growth.

### BACKGROUND

In most cases, male pattern baldness is hereditary with a tendency to greater phenotypical breakthrough from the maternal side. Commonly, male pattern baldness is associated with excessive or otherwise improper 5α-alpha reductase activity in the hair root, and particularly the hair follicle, where the 5α-alpha reductase converts testosterone to dihydrotestosterone (DHT).

Among other suspected activities, DHT is thought to arrest follicular growth patterns in the telogen, probably by reducing blood supply to the hair follicle.

Not surprisingly, numerous attempts have been undertaken to reduce 5α-alpha reductase activity. In one approach, metabolic breakdown of dihydrotestosterone may be stimulated by various compounds in the liver. Alternatively, and more commonly, formation of DHT may be reduced by topical application of Dutasteride or Finasteride, known inhibitors of the 5α-alpha reductase. While both approaches at least conceptually appear attractive, various disadvantages remain. Most significantly, the actual effect of such approaches is typically only very modest (at best), and more commonly fails to significantly improve male pattern baldness.

Therefore, while numerous compositions and methods for stimulation of hair growth are known in the art, all or almost all of them suffer from one or more disadvantages. Consequently, there is still a need to provide improved compositions and methods to stimulate hair growth.

This and all other referenced extrinsic materials are incorporated herein by reference in their entirety. Where a definition or use of a term in an incorporated reference is inconsistent or contrary to the definition of that term provided herein, the definition of that term provided herein applies and the definition of that term in the reference does not apply.

### SUMMARY OF THE INVENTION

The present invention provides a composition for hair growth that includes a lipophilic compound, such as a fatty acid, an allantoin, and a therapeutically effective amount of a biologically active substance.

In preferred embodiments, the fatty acid can be a palmitic, palmitoleic, stearic, oleic, linoleic, linolenic, arachidic, eicosenoic, behenic, erucic, myristic acid, and lignoderic fatty acid. The composition also includes an oil such as jojoba oil, tea tree oil and, wild carrot seed oil. Preferred embodiments also can include soybean oil, canola oil, rapeseed oil, cottonseed oil, coconut oil, palm oil, sesame oil, sunflower oil, safflower oil, rice bran oil, borage seed oil, syzigium aromaticum oil, hempseed oil, herring oil, cod-liver oil, salmon oil, corn oil, flaxseed oil, wheat germ oil, rape seed oil, evening primrose oil, rosehip oil, melaleuca oil and olive oil.

In especially preferred embodiments, the composition further includes a suspended compound which can include: zinc, zinc sulfate, vitamin A, vitamin B complex, vitamin C, vitamin E, alpha hydroxy acid, chromium, copper, and iodine. It also further preferred to include a lubricant, such as silicon and dimethicone in the composition. Preferably, the lipophilic compound is about 5 wt % of the composition and the allantoin is less than and equal than 2 wt %.

It is generally preferred to have a method of promoting hair growth by applying an application of a composition that includes a lipophilic compound, such as a fatty acid, an allantoin, and a therapeutically effective amount of a biologically active substance. Alternatively, the composition can be applied topically in a cream or lotion form to an effective area, such as the head and/or scalp.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

None

### DETAILED DESCRIPTION

The inventor has discovered that specific compositions may address hair loss from at least three different angles by (a) inhibiting DHT production, (b) blocking androgen receptors, and (c) activating hair follicle growth. Remarkably, presently contemplated compositions and methods substantially improve male pattern baldness in a relatively short period of time.

Among other elements, the inventor discovered that specific fatty acids, and especially in combination with allantoin, provide significant effect in hair re-growth. Interestingly, all or most of the contemplated fatty acids are potent inhibitors of 5α-alpha reductase (both type I and II forms up to 98%). Moreover, additional compounds, and especially zinc and vitamin B6 are thought to improve potency of contemplated compositions (probably via inhibition of 5α-alpha reductase).

Most preferably, contemplated formulations include jojoba oil, which is also known to soften skin while being non-greasy. Jojoba oil is generally easily absorbed by the skin, promotes a silky smooth skin, will help nourish the skin, and prevent dryness. Furthermore, jojoba oil is also considered a penetrating odorless oil with a consistency of a liquid wax, making the oil stable and long lasting. A typical chemical analysis of jojoba oil shows it contains palmitic, palmitoleic, stearic, oleic, linoleic, linolenic, and arachidic, eicosenoic, behenic, and erucic and lignoderic fatty acids. It also contains myristic acid, which also has an anti-inflammatory action. Additional beneficial components of jojoba oil include vitamin E and vitamin B complex, silicon, chromium, copper, iodine, and zinc.

Jojoba oil also contains Gamma Linolenic Acid (GLA), Alpha Linolenic Acid (ALA), and Linoleic Acid. These are essential fatty acids found in plant oils. GLA, ALA, and linoleic acid have been individually shown to inhibit 5α-alpha reductase. Remarkably, these fatty acids are among the most powerful inhibitors of 5α-alpha reductase known today and even inhibit the Type I form of the 5α-alpha reductase that is present in high concentrations in the scalp and the skin (inhibited to a significantly lesser degree by finasteride). Oleic acids also present in jojoba oil were reported to have potent anti-inflammatory properties.

Thus, the combination of GLA, ALA, linoleic acid, vitamin B6, zinc sulfate, and copper found in jojoba oil provides a natural yet extremely powerful anti-DHT formulation. These ingredients work through different mechanisms to synergistically inhibit both type 1 and 2 forms of 5α-alpha reductase and decrease DHT locally. By decreasing the DHT levels, hair follicles can grow and thicken naturally, leading to a fuller, healthier scalp without the side effects associated with synthetic drugs.

It is still further preferred that contemplated compositions include tea tree oil, which is obtained by steam distillation of the foliage from the Australian Melaleuca alternifolia tree. Tea tree oil is a natural substance with many uses. It is environmentally safe and has a long history of use and clinical data. Tea tree oil contains over one hundred individual organic compounds. Among other beneficial properties, tea tree oil has a pronounced antifungal and antibacterial effect, which is extremely beneficial for scalp problems. Tea tree oil also helps control fungal dandruff and helps to unblock clogged hair follicles, moisturize the hair and keep the scalp free of bacteria and fungal problems. Tea tree oil, famous for its antiseptic and antifungal properties, has demonstrated the same benefits as five-percent benzoyl peroxide, popular in over-the-counter acne treatments. Tea tree oil is much gentler on the skin. Remarkably, tea tree oil has significant effect on hair re-growth.

Another preferred embodiment contemplates composition including wild carrot seed oil. Wild carrot seed oil contains beta carotene which is rich in Vitamin A and Vitamin C that promote cell generation and blood circulation. The moisturizing effect of wild carrot seed oil allows for the stimulation of the head and scalp for promotion of hair growth. However, other essential oils are also deemed suitable herein, such as soybean oil, canola oil, rapeseed oil, cottonseed oil, coconut oil, palm oil, sesame oil, sunflower oil, safflower oil, rice bran oil, borage seed oil, syzigium aromaticum oil, hempseed oil, herring oil, cod-liver oil, salmon oil, corn oil, flaxseed oil, wheat germ oil, rape seed oil, evening primrose oil, rosehip oil, melaleuca oil and olive oil.

Zinc has been shown to be a beneficial component in the treatment of both alopecia areata and androgenic alopecia. Various studies have suggested that zinc inhibits 5α-alpha reductase, and it is therefore concluded that zinc may be beneficial in diseases and disorders related to excess dihydrotestosterone (DHT) such as androgenetic alopecia, benign prostratic hyperplasia, and acne. Additionally, studies have suggested that vitamin B6, zinc, and azelaic acid combined together, even at very low concentrations, may cause up to 90% inhibition of 5α-alpha reductase activity. Therefore, it is contemplated that zinc may provide a benefit in treating various types of androgen dependent conditions, including androgenetic alopecia. Topical application of zinc has also been shown to reduce sebum production. Zinc in jojoba oil helps promote increase blood flow and has also been shown to have antibacterial effects when applied topically. Copper in jojoba oil helps remove harmful microbes from the hair follicles. Jojoba oil also aids in the removal of impregnated sebum and lowers the pH of the scalp to a healthier, more acidic condition.

In yet further contemplated aspects, the formulations according to the inventive subject matter also contain allantoin. Allantoin is a healing, moisturizing, soothing, and anti-irritating natural component found in rice polishing, earthworms, comfrey root, wheat germ, and as an organic component of many soils. Allantoin has a pronounced keratolytic and skin softening action, and acts as chemical debrider of necrotic and scaling tissue. Allantoin is also used in the treatment of skin ulcers, wounds, bums and sunburns, acne and skin eruptions, abrasions, impetigo, eczema and psoriasis. Allantoin is further known as a valuable cell-proliferating healing agent, which stimulates healthy tissue formation. Where desirable, glycolic acid (or other alpha hydroxy acids) may be added to allantoin acts to demineralize and exfoliate the scalp and follicles and to heal the matrix of the hair surface. Comfrey root from which allantoin is derived provides proper amounts of calcium and phosphorus for strong, healthy hair. Once inside the follicle, allantoin will trigger the formation of new elastic cells, collagen and pigment, filling in cavities of scar tissue. It will also shrink and replace the old fibrous and broken collagen cells. Soon new hair will come through the restored hair follicles in those areas.

The FDA has classified allantoin as a safe and effective active ingredient in skin products. The FDA's Tentative Final Monograph on skin protectant drug products for Over-The-Counter (OCT) human use was published in the Federal Register (Volume 48, No. 32, pp. 6820-33 and Volume 55, No. 11, pp. 25240-81). Based on the wide use and clinical acceptance of allantoin, as well as published reports in the literature, the FDA has approved non-prescription, drug products containing allantoin at 0.5% to 2.0%. Allantoin is considered to be nontoxic, nonirritating and non-allergenic by the Schwartz patch test on 200 individual, as published in the Federal Register (Volume 43, No. 151, pp. 34632-34). It is contemplated that adding allantoin to contemplated compositions as the delivery system further enhances the absorption of the jojoba oil and tea tree oil into the scalp and aids in their delivery to the hair follicle where they can exert their effects.

Additionally, a lubricant, such as silicones and/or dimethicone, may be added to contemplated compositions due to their extremely good lubricity for hair. Even in small amounts they form a protective film around the hair and prevent damage. Because silicone spreads over the hair shaft and displaces water from its surface it enables hair to dry more quickly. Propylene glycol absorbs moisture and aids in thickening hair.

Therefore, in one exemplary aspect of the inventive subject matter, topical compositions for hair re-growth are contemplated that include allantoin in an amount of less or equal than 2 wt % (e.g., 0.5 wt % to 2 wt %), jojoba oil in an amount of about 5 wt % (e.g., 2 wt % to 7 wt %), tea tree oil in an amount of about 1-2 wt %, wild carrot seed oil in an amount of about 1-2 wt %, lanolin oil, dimethicone, and propylene glycol, all of which are formulated in an emollient base. Of course, it should be recognized that additional components, including fragrance, anti-microbial agents, pH stabilizers, etc. may also be included.

Administration of such compositions is preferably topical once to several times daily (or less frequently) in an amount ordinarily used for topical lotions or creams. Depending on the particular composition and condition, administration will provide visible results within a few days after first administration, and it is contemplated that administration may be continued indefinitely, or until a desired amount of hair has grown.

Thus, specific embodiments and applications of compositions and methods related to retroviral propagation have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A composition for hair growth comprising: a lipophilic compound, an allantoin, and a therapeutically effective amount of a biologically active substance.

2. The composition of claim 1, wherein the lipophilic compound comprises at least one fatty acid.

3. The composition of claim 2, wherein the fatty acid is selected from the group consisting of: palmitic, palmitoleic, stearic, oleic, linoleic, linolenic, arachidic, eicosenoic, behenic, erucic, myristic acid, and lignoderic fatty acid.

4. The composition of claim 1, wherein the lipophilic compound includes at least one oil selected from the group consisting of jojoba oil, tea tree oil, wild carrot seed oil, soybean oil, canola oil, rapeseed oil, cottonseed oil, coconut oil, palm oil, sesame oil, sunflower oil, safflower oil, rice bran oil, borage seed oil, syzigium aromaticum oil, hempseed oil, herring oil, cod-liver oil, salmon oil, corn oil, flaxseed oil, wheat germ oil, rape seed oil, evening primrose oil, rosehip oil, melaleuca oil and olive oil.

5. The composition of claim 1, further comprising a suspended compound selected from the group consisting of: zinc, zinc sulfate, vitamin A, vitamin B complex, vitamin C, vitamin E complex, alpha hydroxy acid, chromium, copper, and iodine.

6. The composition of claim 1, further comprising a lubricant.

7. The composition of claim 6, wherein the lubricant is a silicone and/or a dimethicone.

8. The composition of claim 1, wherein the lipophilic compound is about 5 wt %.

9. The composition of claim 1, wherein the allantoin is less than and equal than 2 wt %.

10. A method of promoting hair growth, comprising an application of the composition of claim 1 to an effective area.

11. The method of claim 10, wherein the application is a topical one.

12. The method of claim 10, wherein the application is a lotion.

13. The method of claim 10, wherein the effective area is a head and/or scalp.
